SEQUENCE LISTING (12 sequences)

SEQ ID NO: 1 - DNA, 20 bp, synthetic construct
  source 1..20: mol_type = other DNA; note = forward primer 27F for amplification of 16S rDNA
agagtttgatcctggctcag

SEQ ID NO: 2 - DNA, 18 bp, synthetic construct
  source 1..18: mol_type = other DNA; note = reverse primer 1492R for amplification of 16S rDNA
ggttaccttgttacgact

SEQ ID NO: 3 - DNA, 1380 bp, Bifidobacterium longum
  source 1..1380: mol_type = genomic DNA; note = belongs to the subspecies longum
cgggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaacccgcacccgaagccggtggcctaaccc

SEQ ID NO: 4 - DNA, 1371 bp, Bifidobacterium longum
  source 1..1371: mol_type = genomic DNA; note = belongs to the subspecies longum
cttaccatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccgg

SEQ ID NO: 5 - DNA, 10 bp, synthetic construct
  source 1..10: mol_type = other DNA; note = primer RAPD-1 for RAPD
ctcaggtcgc

SEQ ID NO: 6 - DNA, 10 bp, synthetic construct
  source 1..10: mol_type = other DNA; note = primer RAPD-A for RAPD
ccgcagccaa

SEQ ID NO: 7 - DNA, 10 bp, synthetic construct
  source 1..10: mol_type = other DNA; note = primer RAPD-B for RAPD
aacgcgcaac

SEQ ID NO: 8 - DNA, 10 bp, synthetic construct
  source 1..10: mol_type = other DNA; note = primer RAPD-C for RAPD
gcggaaatag

SEQ ID NO: 9 - DNA, 10 bp, synthetic construct
  source 1..10: mol_type = other DNA; note = primer RAPD-D for RAPD
gaggacaaag

SEQ ID NO: 10 - DNA, 10 bp, synthetic construct
  source 1..10: mol_type = other DNA; note = primer RAPD-E for RAPD
ggcgtcggtt

SEQ ID NO: 11 - DNA, 10 bp, synthetic construct
  source 1..10: mol_type = other DNA; note = primer RAPD-F for RAPD
ggccacggaa

SEQ ID NO: 12 - DNA, 10 bp, synthetic construct
  source 1..10: mol_type = other DNA; note = primer RAPD-G for RAPD
cgaactagac

(19) United States
(12) Patent Application Publication
(10) Pub. No.: US 2026/0124257 A1
(43) Pub. Date: May 7, 2026

(54) BIFIDOBACTERIUM LONGUM STRAIN AND METHOD FOR ALLEVIATING BLUE LIGHT-INDUCED RETINAL DAMAGE USING THE SAME

(71) Applicant: SYNBIO TECH INC., Kaohsiung City (TW)

(72) Inventors: Chi-Hao WU, Taipei City (TW); Han-Yin HSU, Kaohsiung City (TW); Chia-Chia LEE, Kaohsiung City (TW); Jin-Seng LIN, Tainan City (TW)

(21) Appl. No.: 19/291,268

(22) Filed: Aug. 5, 2025

Related U.S. Application Data
(63) Continuation of application No. PCT/CN2025/101738, filed on Jun. 18, 2025.
(60) Provisional application No. 63/716,910, filed on Nov. 6, 2024.

(51) Int. Cl. A61K 35/745; A61P 27/02; C12N 1/20; C12R 1/01

(52) U.S. Cl. CPC A61K 35/745; A61P 27/02; C12N 1/205; C12R 2001/01

(57) ABSTRACT

A Bifidobacterium longum strain is selected from the group consisting of Bifidobacterium longum subsp. longum BL1363 which is deposited at the Deutsche Sammlung von Mikroorganismen und Zellkulturen (DSMZ) GmbH under an accession number DSM 35138, and Bifidobacterium longum subsp. longum BL531 which is deposited at the Deutsche Sammlung von Mikroorganismen und Zellkulturen GmbH under an accession number DSM 35139 in accordance with the Budapest Treaty. A method for alleviating blue light-induced retinal damage is also provided.

### CROSS-REFERENCE TO RELATED APPLICATION

This application is a continuation application of International Application No. PCT/CN2025/101738, filed on Jun. 18, 2025, which claims priority to U.S. Provisional Patent Application No. 63/716,910, filed on Nov. 6, 2024. The aforesaid applications are incorporated by reference herein in their entirety.

### SEQUENCE LISTING XML

The Sequence Listing submitted concurrently herewith with a file name of “Substitute_Sequence_Listing.xml” a creation date of Sep. 25, 2025, and a size of 16,384 bytes, is part of the specification and is incorporated by reference in its entirety

### FIELD

The disclosure relates to a Bifidobacterium longum strain, which is selected from the group consisting of Bifidobacterium longum subsp. longum BL1363 and Bifidobacterium longum subsp. longum BL531, and a method for alleviating blue light-induced retinal damage using the same.

### BACKGROUND

Blue light is a high-energy visible light, and exposure to blue light too frequently may cause a retina of an eye to be damaged (i.e., retinal damage).

At present, there is no clinically effective method to alleviate blue light-induced retinal damage. The only way to avoid such damage is to reduce exposure to blue light sources (e.g., computers, communication devices, and consumer electronics), and to consume a health supplement that is beneficial to eyes, such as lutein and fish oil.

Lactic acid bacteria (LAB) are generally recognized as safe (GRAS), well-known and widely used probiotics, and have been found to have the effects of inhibiting the growth of gastrointestinal pathogens, alleviating lactose intolerance, anti-cancer and lowering blood pressure. There are many types of lactic acid bacteria that can be used as probiotics, such as Lactobacillus, Lactococcus, Pediococcus, Enterococcus, Streptococcus, and Bifidobacterium.

In spite of the aforesaid, there is still a need to develop a method that is effective in alleviation of blue light-induced retinal damage.

### SUMMARY

Accordingly, in a first aspect, the present disclosure provides a Bifidobacterium longum strain, which can alleviate at least one of the drawbacks of the prior art, and which is selected from the group consisting of Bifidobacterium longum subsp. longum BL1363 which is deposited at the Deutsche Sammlung von Mikroorganismen und Zellkulturen (DSMZ) GmbH under an accession number DSM 35138, and Bifidobacterium longum subsp. longum BL531 which is deposited at the Deutsche Sammlung von Mikroorganismen und Zellkulturen GmbH under an accession number DSM 35139 in accordance with the Budapest Treaty.

In a second aspect, the present disclosure provides a method for alleviating blue light-induced retinal damage, which can alleviate at least one of the drawbacks of the prior art, and which includes administering to a subject in need thereof a composition containing a Bifidobacterium longum strain. The Bifidobacterium longum strain is selected from the group consisting of Bifidobacterium longum subsp. longum BL1363 which is deposited at the Deutsche Sammlung von Mikroorganismen und Zellkulturen (DSMZ) GmbH under an accession number DSM 35138, Bifidobacterium longum subsp. longum BL531 which is deposited at the Deutsche Sammlung von Mikroorganismen und Zellkulturen GmbH under an accession number DSM 35139 in accordance with the Budapest Treaty, and a combination thereof.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows genetic fingerprints of each of a conventional Bifidobacterium longum subsp. longum DSM 20219^{T} (Lane 1), and Bifidobacterium longum subsp. longum BL531 (Lane 2) and Bifidobacterium longum subsp. longum BL1363 (Lane 3) according to the present disclosure, infra, which are respectively obtained by random amplified polymorphic DNA (RAPD) analysis using different 10-mer primers, and in which Lane M represents a DNA ladder marker (100 bp to 3000 bp).

FIG. 2 shows the visual acuity threshold of the mice in each group of Example 2 determined at the end of each of the 8^{th} week and the 16^{th} week after starting irradiation with blue light, infra, in which the data of different groups in each of the 8^{th} week and the 16^{th} week are marked with different English letters to indicate statistical differences therebetween.

FIG. 3 shows the retinal potential changes of the mice in each group of Example 2 determined at the end of each of the 8^{th} week and the 16^{th} week after starting the irradiation with the blue light, infra, in which the data of different groups for each of the a-wave and the b-wave measured in each of the 8^{th} week and the 16^{th} week are marked with different English letters to indicate statistical differences therebetween.

FIG. 4 shows the result of histopathological analysis of the mice in each group of Example 2 determined at the end of the 16^{th} week after starting the irradiation with the blue light, infra, in which “GCL” represents a ganglion cell layer, “INL” represents an inner nuclear layer, “ONL” represents an outer nuclear layer, “IS/OS” represents an inner segment/outer segment layer, and “RPE” represents a retinal pigment epithelium.

FIG. 5 shows the number of nuclei in the ONL of the mice in each group of Example 2 determined at the end of the 16^{th} week after starting the irradiation with the blue light, infra, in which the data of different groups are marked with different English letters to indicate statistical differences therebetween.

### DETAILED DESCRIPTION

It is to be understood that, if any prior art publication is referred to herein, such reference does not constitute an admission that the publication forms a part of the common general knowledge in the art.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of approaches to alleviate blue light-induced retinal damage, the applicant surprisingly found that two strains of Bifidobacterium longum subsp. longum isolates, which were respectively named “Bifidobacterium longum subsp. longum BL1363” and “Bifidobacterium longum subsp. longum BL531” by the applicant, and deposited at the Deutsche Sammlung von Mikroorganismen und Zellkulturen GmbH under an accession number DSM 35138 and an accession number DSM 35139, respectively, have been demonstrated through in vivo experiments to be capable of effectively alleviating decreased visual acuity, retinal dysfunction and retinopathy caused by blue light exposure, and hence are expected to have high potential to be developed into drugs for use in alleviation of blue light-induced retinal damage.

Therefore, the present disclosure provides a Bifidobacterium longum strain, which is selected from the group consisting of Bifidobacterium longum subsp. longum BL1363 which is deposited at the Deutsche Sammlung von Mikroorganismen und Zellkulturen (DSMZ) GmbH under an accession number DSM 35138, and Bifidobacterium longum subsp. longum BL531 which is deposited at the Deutsche Sammlung von Mikroorganismen und Zellkulturen GmbH under an accession number DSM 35139 in accordance with the Budapest Treaty.

The present disclosure also provides a method for alleviating blue light-induced retinal damage, comprising administering to a subject in need thereof a composition containing a Bifidobacterium longum strain, wherein the Bifidobacterium longum strain is selected from the group consisting of Bifidobacterium longum subsp. longum BL1363 which is deposited at the Deutsche Sammlung von Mikroorganismen und Zellkulturen GmbH under an accession number DSM 35138, Bifidobacterium longum subsp. longum BL531 which is deposited at the Deutsche Sammlung von Mikroorganismen und Zellkulturen GmbH under an accession number DSM 35139 in accordance with the Budapest Treaty, and a combination thereof.

In certain embodiments, the term “alleviating blue light-induced retinal damage” means that at least one of the following therapeutic efficacy is achieved: enhancing visual acuity, improving retinal function, and alleviating retinopathy.

As used herein, the terms “administering” and “administration” can be interchangeably used, and refer to introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human. In other embodiments, the subject does not have dry eye syndrome.

In certain embodiments, the Bifidobacterium longum strain is the combination of Bifidobacterium longum subsp. longum BL1363 and Bifidobacterium longum subsp. longum BL531. In certain embodiments, a number ratio of Bifidobacterium longum subsp. longum BL1363 to Bifidobacterium longum subsp. longum BL531 may range from 1:1 to 1:5.

According to the present disclosure, the Bifidobacterium longum strain may be live cells or dead cells, concentrated or non-concentrated, a liquid, a paste, a semi-solid, or a solid (e.g., a pellet, a granule, or a powder), and may be heat-inactivated, frozen, dried, or freeze-dried (e.g., may be in freeze-dried form or spray/fluid bed dried form). In an exemplary embodiment, the Bifidobacterium longum strain is in the form of live cells.

According to the present disclosure, the Bifidobacterium longum strain may be prepared into a bacterial liquid having a total bacterial concentration ranging from 1×10⁶ CFU/mL to 1×10⁹ CFU/mL. In an exemplary embodiment, the bacterial liquid has a total bacterial concentration of 3.3×10⁸ CFU/mL.

According to the present disclosure, the culture medium suitable for culturing the Bifidobacterium longum strain may be prepared using techniques well-known to those skilled in the art, or may be commercially available products, which may include, but are not limited to, MRS (De Man, Rogosa and Sharpe) broth, MRS broth supplemented with cysteine, and a mineral medium supplemented with glucose and soy peptone. In an exemplary embodiment, the culture medium is the MRS broth.

As used herein, the terms “culturing”, “fermentation” and “cultivation” may be used interchangeably.

The procedures and parameter conditions for cultivation are within the expertise and routine skills of those skilled in the art, and may be adjusted according to practical requirements.

According to the present disclosure, the cultivation may be carried out at a temperature ranging from 30° C. to 37° C. for a time period ranging from 16 hours to 18 hours. In an exemplary embodiment, the cultivation is carried out at 37° C. for the time period ranging from 16 hours to 18 hours.

According to the present disclosure, the composition may be formulated as a food product using a standard technique well known to one of ordinary skill in the art. For instance, the aforesaid composition may be directly added to an edible material, or may be utilized for preparing an intermediate composition (e.g., a food additive or a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, and dietary supplements.

According to the present disclosure, the composition may be prepared in the form of a pharmaceutical composition.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the pharmaceutical composition may be formulated into a dosage form suitable for oral administration or parenteral administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intralesional injection and sublingual administration.

According to the present disclosure, the dose and frequency of administration of the composition may vary depending on the following factors: the severity of the illness or disorder to be treated, routes of administration, and age, physical condition and response of the subject to be treated. In general, the composition may be administered orally or parenterally in a single dose or in several doses.

The present disclosure also provides the aforesaid composition for use in alleviation of blue light-induced retinal damage. The use includes administering to a subject in need thereof the aforesaid composition containing the Bifidobacterium longum strain.

The present disclosure further provides use of the aforesaid composition in the manufacture of a medication or a food product in alleviation of blue-light induced retinal damage in a subject.

The disclosure will be further described by way of the following examples. However, it should be understood that the following examples are solely intended for the purpose of illustration and should not be construed as limiting the disclosure in practice.

### Examples

### General Experimental Materials:

### 1. Experimental Mice:

Male ICR mice (8 weeks old) used in the following experiments were purchased from BioLasco Taiwan Co., Ltd. All the experimental mice were individually housed in an animal room under the following laboratory conditions: an alternating 12-hour light and 12-hour dark cycle, a temperature maintained at 22° C.±2° C., a relative humidity maintained at 60%±10%. Furthermore, water and feed were provided ad libitum for all the experimental mice. All the experimental mice were kept for 2 weeks before the experiments in order to adapt to the environment. All experimental procedures involving the experimental mice were approved by the Institutional Animal Care and Use Committees in China Taiwan Normal University, and were carried out in accordance with the Guide for the Care and Use of Laboratory Animals of National Institutes of Health (NIH).

### General Procedures:

### 1. Statistical Analysis:

The experimental data of all the test groups are expressed as mean±standard deviation (SD), and were analyzed using one-way analysis of variance (one-way ANOVA) followed by post hoc test, so as to evaluate the differences between the groups. Statistical significance is indicated by p<0.05.

### Example 1. Screening and Identification of Isolates BL1363 and BL531

### A. Source and Isolation of Isolates BL1363 and BL531:

Feces were collected from healthy humans aged over 85 years old and had not used antibiotics for 3 months to serve as sample sources for isolation and screening of Bifidobacteria. First, the feces were mixed with an appropriate amount of saline solution (added with 0.05% of L-Cysteine) to obtain a mixture, and then the mixture was subjected to homogenization using a homogenizer (Scientific Industries, Inc.; Model: Vortex-Genie 2). Next, the homogenate thus obtained was subjected to 10-fold serial dilution with a saline solution, so that 10⁻¹-fold to 10⁻⁶-fold diluents were obtained. Subsequently, 0.1 mL of each of the 10⁻¹-fold to 10⁻⁶-fold diluents was taken and then evenly spread on MRS agar medium, followed by cultivation in an incubator (37° C.) under an anaerobic condition for 5 days to 7 days. The colonies formed on the MRS agar medium were picked to obtain two isolated strains, i.e., isolates BL1363 and BL531.

### B. Preliminary Tests:

The isolates BL1363 and BL531 were subjected to Gram staining, and the results showed that the isolates BL1363 and BL531 were both Gram-positive bacilli.

### C. Extraction of Genomic DNA:

The isolates BL1363 and BL531 were subjected to extraction of genomic DNA conducted generally according to the method described in Zhu, H et al. (1993), Nucleic Acids Res., 21(22):5279-5280, in which an extraction buffer (containing 100 mM Tris and 40 mM EDTA; pH 9.0), Mini-BeadBeater Glass Mill Beads (0.1 mm) (BioSpec Products; Cat. No. 11079101), and 500 μL of phenol-chloroform-isoamyl alcohol mixture (25:24:1, v/v/v) were used.

D. 16S Ribosomal DNA (rDNA) Sequence Analysis:

The resultant genomic DNA of each of the isolates BL1363 and BL531 obtained in Section C of this example, serving as a template, was subjected to polymerase chain reaction (PCR) performed using a designed primer pair specific for bacterial 16S rDNA gene shown in Table 1 under the reaction conditions shown in Table 2 below, thereby obtaining a PCR product (i.e., PCR-amplified 16S rDNA fragments) of the same.

TABLE 1

| | Nucleotide sequence |
|---|---|
| PCR primer pair | (5′ -> 3′) |
| Forward primer | agagtttgatcctggctcag |
| 27F | (SEQ ID NO: 1) |
| Reverse primer | ggttaccttgttacgact |
| 1492R | (SEQ ID NO: 2) |

TABLE 2

| Reaction mix | Volume (μL) |
|---|---|
| Genomic DNA (10 ng/μL) | 1 |
| Forward primer 27F (10 μM) | 0.5 |
| Reverse primer 1492R (10 μM) | 0.5 |
| Fast-Run ™ 2X Tag Master Mix | 10 |
| (Protech, Cat. No. SA-TMM228) | |
| Deionized water | 8 |

Operation conditions: denaturation at 95° C. for 5 minutes, followed by 30 cycles of the following reactions: denaturation at 95° C. for 60 seconds, primer annealing at 50° C. for 60 seconds, extension at 72° C. for 60 seconds; and finally, elongation at 72° C. for 8 minutes

Thereafter, the PCR product was verified by sequencing analysis which was entrusted to Genomics BioSci & Tech Co., Ltd., so as to obtain a part of 16S rDNA sequence of each of the isolates BL1363 (SEQ ID No: 3) and BL531 (SEQ ID No: 4), respectively. By comparing the data in the NCBI's gene database, the part of the 16S rDNA sequence of each of the isolates BL1363 and BL531 is found to have 97% identity to a part of the 16S rDNA sequence of Bifidobacterium longum subsp. longum.

In view of the aforesaid experimental results, the isolates BL1363 and BL531 of the present disclosure are identified as Bifidobacterium longum subsp. longum. In order to confirm whether each of Bifidobacterium longum subsp. longum BL1363 and Bifidobacterium longum subsp. longum BL531 is a novel Bifidobacterium longum subsp. longum strain, the following experiment was conducted.

### E. Random Amplified Polymorphic DNA (RAPD) Analysis:

The RAPD analysis was generally performed with reference to Akopyanz N. et al. (1992), Nucleic Acids Res., 20:5137-5142. Briefly, the resultant genomic DNA of each of the isolates BL1363 and BL531 obtained in Section C of this example, serving as a template, was subjected to polymerase chain reaction (PCR) performed using eight 10-mer primers shown in Table 3 under the reaction conditions shown in Table 4 below.

TABLE 3

| | Nucleotide sequence |
|---|---|
| Primer | (5′ -> 3′) |
| RAPD-1 | ctcaggtcgc (SEQ ID NO: 5) |
| RAPD-A | ccgcagccaa (SEQ ID NO: 6) |
| RAPD-B | aacgcgcaac (SEQ ID NO: 7) |
| RAPD-C | gcggaaatag (SEQ ID NO: 8) |
| RAPD-D | gaggacaaag (SEQ ID NO: 9) |
| RAPD-E | ggcgtcggtt (SEQ ID NO: 10) |
| RAPD-F | ggccacggaa (SEQ ID NO: 11) |
| RAPD-G | cgaactagac (SEQ ID NO: 12) |

TABLE 4

| Reaction mix | Volume (μL) |
|---|---|
| Genomic DNA (50-100 ng) | 1 |
| 10-mer primer (10 μM) | 4 |
| 2X Taq Master Mix | 12.5 |
| (Ampliqon, 5200300-1250) | |
| MgCl₂ (25 mM) | 2 |
| Deionized water | 5.5 |

Operation conditions: denaturation at 94° C. for 2 minutes, followed by 6 cycles of the following reactions: denaturation at 94° C. for 30 seconds, primer annealing at 36° C. for 60 seconds, and extension at 72° C. for 90 seconds, and then by 30 cycles of the following reactions: denaturation at 94° C. for 20 seconds, primer annealing at 36° C. for 30 seconds, extension at 72° C. for 90 seconds; and finally, elongation at 72° C. for 8 minutes

The resultant PCR product was subjected to 1.5% agarose gel electrophoresis analysis, followed by staining, and then observation and photography under ultraviolet light.

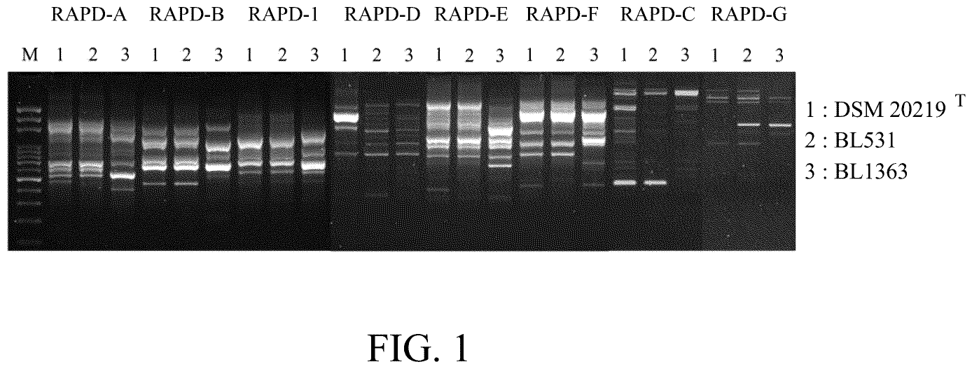

For comparison purpose, the applicant used a conventional Bifidobacterium longum subsp. longum DSM 20219^{T} (corresponding to Bifidobacterium longum subsp. longum BCRC 11847^{T}) as a comparative strain to be compared with Bifidobacterium longum subsp. longum BL1363 and Bifidobacterium longum subsp. longum BL531. The results of the 1.5% agarose gel electrophoresis analysis were shown in FIG. 1.

The results in FIG. 1 show that the genetic fingerprints of Bifidobacterium longum subsp. longum BL1363, Bifidobacterium longum subsp. longum BL531, and the conventional Bifidobacterium longum subsp. longum DSM 20219^{T} were different from each other.

Based on the aforementioned characterization results, the applicant believes that each of Bifidobacterium longum subsp. longum BL1363 and Bifidobacterium longum subsp. longum BL531 of the present disclosure is a novel strain of Bifidobacterium longum subsp. longum. As such, Bifidobacterium longum subsp. longum BL1363 and Bifidobacterium longum subsp. longum BL531 have been deposited at the Bioresource Collection and Research Center (BCRC) of the Food Industry Research and Development Institute (FIRDI) (No. 331, Shih-Pin Rd., Hsinchu City 300, Taiwan, China) respectively under an accession number BCRC 911238 and an accession number BCRC 911239 since Aug. 28, 2024, and have also been deposited at the Deutsche Sammlung von Mikroorganismen und Zellkulturen (DSMZ) GmbH respectively under an accession number DSM 35138 and an accession number DSM 35139 since Sep. 9, 2024 in accordance with the Budapest Treaty.

### Example 2. Evaluation of the Efficacy of Bifidobacterium longum Subsp. longum BL1363 and Bifidobacterium longum Subsp. longum BL531 in Alleviating Blue Light-Induced Retinal Damage

### Experimental Materials:

1. Preparation of Bacterial Suspension of Bifidobacterium longum Subsp. Longum BL1363 and Bifidobacterium longum Subsp. Longum BL531:

A respective one of Bifidobacterium longum subsp. longum BL1363 and Bifidobacterium longum subsp. longum BL531 was inoculated into MRS broth medium (BD Difco™; Cat. No. 288130), and then cultured at 37° C. for 16 hours to 18 hours for activation of the same. After centrifugation at 5000 rpm and 4° C. for 10 minutes, the resultant cell pellet and the supernatant were formed. The supernatant was then collected, and the cell pellet was washed with an appropriate amount of phosphate-buffered saline (PBS), followed by using an appropriate amount of the supernatant to re-suspend the cell pellet (the appropriate amount of the supernatant was also used to adjust bacterial concentration of the subsequently obtained bacterial suspension so as to have a predetermined value), thereby obtaining bacterial suspensions of Bifidobacterium longum subsp. longum BL1363 and Bifidobacterium longum subsp. longum BL531, each having a bacterial concentration of 3.3×10⁸ CFU/mL.

### Experimental Procedures and Results:

### A. Administration of Bacterial Suspension and Induction of Retinal Damage:

First, the male ICR mice were randomly divided into 4 groups (n=10 in each group), including one normal control group, one pathological control group, and two experimental groups (i.e., experimental groups 1 and 2). After that, each of the mice in the experimental group 1 was fed, via oral gavage, with the bacterial suspension of Bifidobacterium longum subsp. longum BL1363 (dose: 1×10⁸ CFU/mouse) obtained in Section 1 of Experimental Materials of this example, and each of the mice in the experimental group 2 was fed, via oral gavage, with the bacterial suspension of Bifidobacterium longum subsp. longum BL531 (dose: 1×10⁸ CFU/mouse) obtained in Section 1 of Experimental Materials of this example. Each of the mice in the pathological control group and the normal control group was fed, via oral gavage, with MRS broth medium having a volume equal to that of the bacterial suspension of Bifidobacterium longum subsp. longum BL1363 or Bifidobacterium longum subsp. longum BL531. Each mouse was fed once a day for a time period of 17 weeks.

From the 2^{nd} week after starting administration of the bacterial suspensions or the MRS broth medium, the mice in the pathological control group and the experimental groups 1 and 2 were subjected to irradiation with blue light having a wavelength of 465±10 nm and an illumination of 108 lux (44.8 μW/cm²) twice a day, 3 hours each time, for a time period of 16 weeks. The mice in the normal control group received no treatment.

### B. Visual Acuity Testing:

At the end of each of the 8^{th} week and the 16^{th} week after starting the irradiation with the blue light, the mice in each group were subjected to visual acuity testing conducted generally according to the method described in Liou J. C. et al. (2018), J. Funct. Foods, 49:314-323. Specifically, each of the mice in each group was placed on a cylindrical platform located in the center of a dark box with screens on four sides thereof, and then gratings respectively having spatial frequencies of 0.437 cycles per degree (cpd), 0.328 cpd, 0.164 cpd, 0.082 cpd, 0.055 cpd, and 0.033 cpd were played on the screens in sequence. Each of the special frequencies (i.e., each of the gratings) lasted for 2 minutes, during which such grating moved alternately in clockwise and counterclockwise directions at a rotation speed of 12°/s. Next, the visual acuity threshold was obtained by observing at which of the aforesaid spatial frequency the mice's head could move in the direction of grating movement with an accuracy rate of more than 70%.

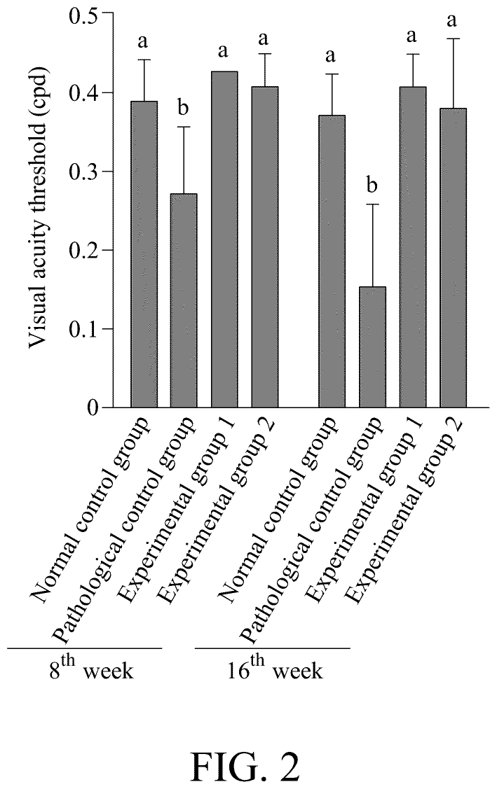

The data thus obtained were analyzed according to the procedures as described in Section 1 of the General Procedures. The results were shown in FIG. 2.

Referring to FIG. 2, at the end of the 8^{th} week after starting the irradiation with the blue light, the visual acuity threshold determined in the pathological control group showed a significant decrease compared with the visual acuity threshold determined in the normal control group, indicating that the blue light successfully induced a decrease in visual acuity in the mice of the pathological control group. In comparison with the pathological control group, the visual acuity threshold determined in each of experimental groups 1 and 2 was significantly increased and was close to the visual acuity threshold determined in the normal control group. At the end of the 16^{th} week after starting the irradiation with the blue light, similar results could be observed. These results show that both Bifidobacterium longum subsp. longum BL1363 and Bifidobacterium longum subsp. longum BL531 of the present disclosure can effectively alleviate the decrease in visual acuity caused by blue light irradiation.

### C. Analysis of Electroretinogram (ERG):

At the end of each of the 8^{th} week and the 16^{th} week after starting the irradiation with the blue light, the mice in each group were placed in a dark environment for 12 hours and then anesthetized with Xylazine (dose: 9.75 mg/kg) and Zoletil 100 (dose: 75 mg/kg). Next, each mouse was subjected to mydriasis by instilling one to two drops of 0.5% Tropicamide in a cornea thereof, and then subjected to analysis of retinal potential using BPM-300 electrodiagnostic system (version of software: 7.0.0.7) (RetinoGraphics) as follows. First, a reference electrode and an indifferent electrode were connected to an outer corner of an eye and a forehead of the mouse, respectively, and a recording electrode was attached to a surface of the cornea. The resistance value of each of the recording electrode and the reference electrode was maintained at 2.1 kΩ, and the resistance value of the indifferent electrode was maintained at 12.1±3.025 kΩ. A white light having a brightness of 3 cd/m² and a flickering frequency of once per second was used to stimulate a retina of the mouse at a distance of approximately 3 mm to 5 mm from the cornea of the mouse, so as to produce retinal potential changes, followed by recording a-wave and b-wave.

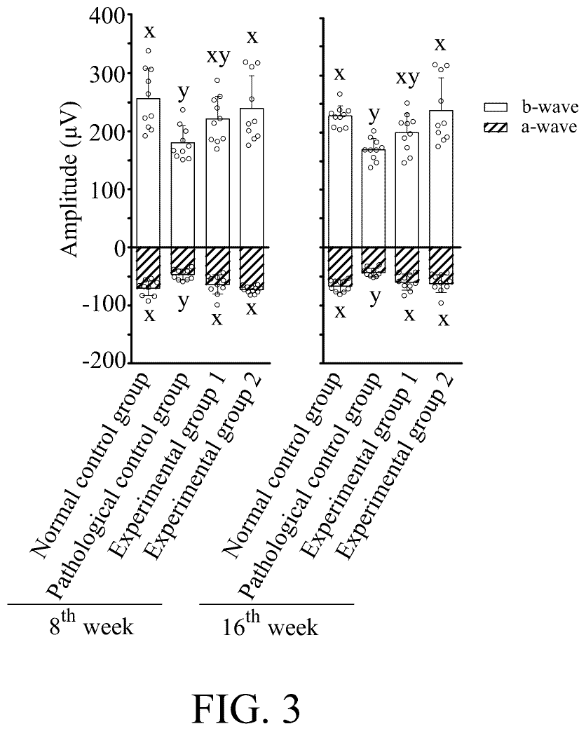

The data thus obtained were analyzed according to the procedures as described in Section 1 of the General Procedures. The results were shown in FIG. 3.

Referring to FIG. 3, at the end of the 8^{th} week after starting the irradiation with the blue light, for each of the a-wave and the b-wave, the retinal potential determined in the pathological control group showed a significant decrease compared with the retinal potential determined in the normal control group, indicating that the blue light successfully induced retinal dysfunction in the mice of the pathological control group. In addition, for each of the a-wave and the b-wave, the retinal potential determined in each of experimental groups 1 and 2 was significantly increased compared with the retinal potential determined in the pathological control group. There was no significant difference between the retinal potential determined in each of experimental groups 1 and 2 and that determined in the normal control group. At the end of the 16^{th} week after starting the irradiation with the blue light, similar results could be observed. These results demonstrate that both Bifidobacterium longum subsp. longum BL1363 and Bifidobacterium longum subsp. longum BL531 of the present disclosure can effectively alleviate retinal dysfunction caused by blue light irradiation.

### D. Histopathological Analysis:

At the end of the 16^{th} week after starting the irradiation of the blue light, the mice in each group were anesthetized with 3% to 4% isoflurane and sacrificed, and then eyeballs thereof were removed and subjected to a fixation treatment using a fixative solution (containing 347 mL of ddH₂O, 111 mL 100% acetic acid, 320 mL of 99.5% ethanol and 222 mL of 10% formaldehyde) overnight, followed by a dehydration treatment using ethanol. The dehydrated eyeballs were then embedded with paraffin, followed by slicing, so as to obtain a retinal tissue section having a thickness of 4 μm.

Thereafter, the retinal tissue section was subjected to hematoxylin-eosin staining using a staining protocol well-known to those skilled in the art, and was then observed and photographed under an optical microscope (Olympus; Model: CX33) at a magnification of 40×, followed by using ViewPoint software to count the number of nuclei in an outer nuclear layer (ONL).

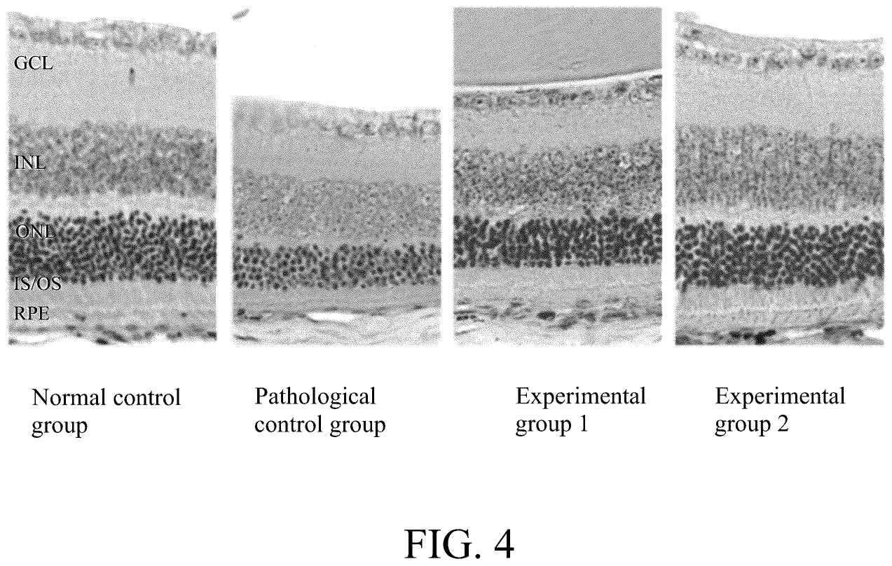

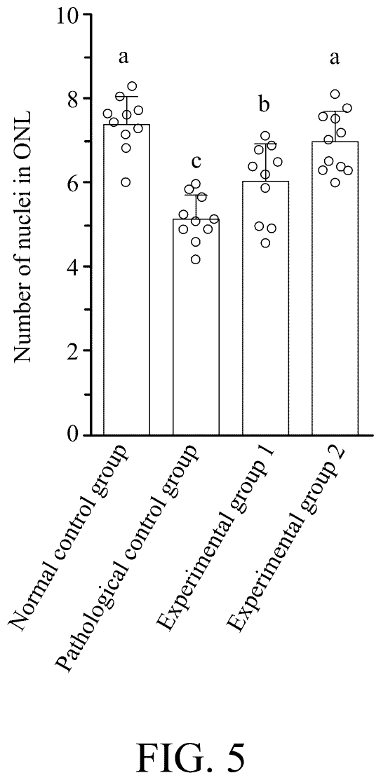

The data thus obtained were analyzed according to the procedures as described in Section 1 of the General Procedures. The results were shown in FIGS. 4 and 5.

Referring to FIG. 4, compared with the normal control group, the thickness of the ONL and the thickness of an inner segment/outer segment layer (IS/OS) of the pathological control group showed a significant decrease after the mice were subjected to the irradiation of the blue light, indicating that the blue light successfully induced retinopathy in the mice of the same. In comparison with the pathological control group, the thickness of the ONL and the thickness of the IS/OS of each of the experimental groups 1 and 2 were significant increased and were respectively similar to those of the normal control group.

Referring to FIG. 5, compared with the normal control group, the number of nuclei in the ONL determined in the pathological control group was significantly reduced after the mice were subjected to the irradiation of the blue light, indicating that the blue light successfully induced retinopathy in the mice of the pathological control group. In comparison with the pathological control group, the number of nuclei in the ONL determined in each of the experimental groups 1 and 2 was significantly increased, and the number of nuclei in the ONL determined in the experimental group 2 was similar to that determined in the normal control group.

These results demonstrate that both Bifidobacterium longum subsp. longum BL1363 and Bifidobacterium longum subsp. longum BL531 of the present disclosure can effectively alleviate the retinopathy caused by blue light irradiation and slow down the loss of retinal tissue.

In sum, Bifidobacterium longum subsp. longum BL1363 and Bifidobacterium longum subsp. longum BL531 of the present disclosure can exhibit an excellent effect in alleviating blue light-induced retinal damage.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for alleviating blue light-induced retinal damage, comprising administering to a subject in need thereof a composition containing a Bifidobacterium longum strain, wherein the Bifidobacterium longum strain is selected from the group consisting of Bifidobacterium longum subsp. longum BL1363 which is deposited at the Deutsche Sammlung von Mikroorganismen und Zellkulturen (DSMZ) GmbH under an accession number DSM 35138, Bifidobacterium longum subsp. longum BL531 which is deposited at the Deutsche Sammlung von Mikroorganismen und Zellkulturen GmbH under an accession number DSM 35139 in accordance with the Budapest Treaty, and a combination thereof.

2. The method as claimed in claim 1, wherein the Bifidobacterium longum strain is the combination of Bifidobacterium longum subsp. longum BL1363 and Bifidobacterium longum subsp. longum BL531.

3. The method as claimed in claim 2, wherein a number ratio of Bifidobacterium longum subsp. longum BL1363 to Bifidobacterium longum subsp. longum BL531 ranges from 1:1 to 1:5.

4. The method as claimed in claim 1, wherein the composition is a food product.

5. The method as claimed in claim 1, wherein the composition is a pharmaceutical composition.

6. The method as claimed in claim 5, wherein the pharmaceutical composition further contains a pharmaceutically acceptable carrier.

7. The method as claimed in claim 5, wherein the pharmaceutical composition is in a dosage form selected from the group consisting of an oral dosage form and a parenteral dosage form.
